(19) United States
(12) Patent Application Publication
(10) Pub. No.: US 2006/0116538 A1
(43) Pub. Date: Jun. 1, 2006

(54) Azeotrope compositions comprising 1,1,3,3,3-pentafluoropropene and hydrogen fluoride and uses thereof

(76) Inventors: Ralph Newton Miller, Newark, DE (US); Mario Joseph Nappa, Newark, DE (US); Velliyur Nott Mallikarjuna Rao, Wilmington, DE (US); Allen Capron Sievert, Elkton, MD (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/264,209

(22) Filed: Nov. 1, 2005

Related U.S. Application Data
(63) Continuation-in-part of application No. 11/259,901, filed on Oct. 27, 2005.
(60) Provisional application No. 60/623,210, filed on Oct. 29, 2004.

(51) Int. Cl. C07C 17/38

(52) U.S. Cl. 570/178

(57) ABSTRACT

Disclosed herein are azeotrope and near-azeotrope compositions comprising 1,1,3,3,3-pentafluoropentene (HFC-1225zc) and hydrogen fluoride. These compositions are useful in processes to produce and purify HFC-1225zc. Also disclosed are processes comprising pyrolyzing CF₃CH₂CF₃ to CF₃CH═CF₂. Also disclosed herein are processes for the manufacture of HFC-1225zc comprising pyrolyzing 1,1,1,3,3,3-hexafluoropropane (HFC-236fa) in the absence of dehydrofluorination catalyst in a reactor having a reaction zone.

### CROSS-REFERENCE TO RELATED APPLICATIONS

This application is a continuation-in-part of U.S. Patent Application No. ______ (Attorney Docket No. FL 0290 US NA) filed Oct. 27, 2005, which claims the benefit of Provisional U.S. Patent Application No. 60/623,210 filed Oct. 29, 2004, both of which are incorporated herein by reference in their entireties.

### BACKGROUND OF THE INVENTION

1. Field of the Invention

Disclosed herein are azeotrope compositions comprising 1,1,3,3,3-pentafluoropropene and hydrogen fluoride. The azeotrope compositions are useful in processes to produce and in processes to purify 1,1,3,3,3-pentafluoropropene. The present disclosure also relates to processes for the manufacture of HFC-1225zc comprising pyrolyzing 1,1,1,3,3,3-hexafluoropropane (HFC-236fa) in the absence of dehydrofluorination catalyst in a reactor having a reaction zone.

2. Description of Related Art

Chlorine-containing compounds, such as chlorofluorocarbons (CFCs) are considered to be detrimental to the Earth's ozone layer. Many of the hydrofluorocarbons (HFCs), used to replace CFCs, have been found to contribute to global warming. Therefore, there is a need to identify new compounds that do not damage the environment, but also possess the properties necessary to function as refrigerants, solvents, cleaning agents, foam blowing agents, aerosol propellants, heat transfer media, dielectrics, fire extinguishing agents, sterilants and power cycle working fluids. Fluorinated olefins, containing one or more hydrogens in the molecule, are being considered for use in some of the applications, like for example in refrigeration.

1,1,3,3,3-Pentafluoropropene is a useful cure-site monomer in polymerizations to form fluoroelastomers. U.S. Pat. Nos. 6,703,533, 6,548,720, 6,476,281, 6,369,284, 6,093,859, and 6,031,141, as well as published Japanese patent applications JP 09095459 and JP 09067281, and WIPO publication WO 2004018093, disclose processes wherein 1,1,1,3,3,3-hexafluoropropane is heated at temperatures below 500° C. in the presence of catalyst to form 1,1,3,3,3-pentafluoropropene. These low-temperature catalytic routes are chosen because of the well-known tendency for fluorocarbons to fragment at higher temperatures, e.g., above 500° C. This is made clear in Chemistry of Organic Fluorine Compounds, by Milos Hudlicky, 2^{nd} Revised Edition, Ellis Horwood PTR Prentice Hall [1992] p. 515: “Polyfluoroparaffins and especially fluorocarbons and other perfluoro derivates show remarkable heat stability. They usually do not decompose at temperatures below 300° C. Intentional decomposition, however, carried out at temperatures of 500-800° C., causes all possible splits in their molecules and produces complex mixtures which are difficult to separate.”

U.S. Patent Application Publication 2002/0032356 discloses a process for producing the perfluorinated monomers tetrafluoroethylene and hexafluoropropylene in a gold-lined pyrolysis reactor.

The catalytic process has disadvantages, including catalyst preparation, start-up using fresh catalyst, catalyst deactivation, potential for plugging of catalyst-packed reactors with polymeric by-products, catalyst disposal or reactivation, and long reaction times that impose a space/time/yield reactor penalty.

### BRIEF SUMMARY OF THE INVENTION

One aspect relates to an azeotrope or near-azeotrope composition comprising 1,1,3,3,3-pentafluoropropene (HFC-1225zc) and hydrogen fluoride (HF).

A further aspect relates to a process for the separation of HFC-1225zc from 1,1,1,3,3,3-hexafluoropropane (HFC-236fa) comprising: a) forming a mixture of HFC-1225zc, HFC-236fa, and hydrogen fluoride; and b) subjecting said mixture to a distillation step forming a column distillate composition comprising an azeotrope or near-azeotrope composition of hydrogen fluoride and HFC-1225zc essentially free of HFC-236fa.

A further aspect relates to a process for the separation of HFC-1225zc from a mixture comprising an azeotrope or near-azeotrope composition of HFC-1225zc and hydrogen fluoride, said process comprising: a) subjecting said mixture to a first distillation step in which a composition enriched in either (i) hydrogen fluoride or (ii) HFC-1225zc is removed as a first distillate composition with a first bottoms composition being enriched in the other of said components (i) or (ii); and b) subjecting said first distillate composition to a second distillation step conducted at a different pressure in which the component enriched as first bottoms composition in (a) is removed in a second distillate composition with a second bottoms composition enriched in the same component which was enriched in the first distillate composition.

A further aspect relates to a process for the purification of HFC-1225zc from a mixture of HFC-1225zc, HFC-236fa, and hydrogen fluoride, said process comprising: a) subjecting said mixture to a first distillation step to form a first distillate comprising an azeotrope or near-azeotrope composition containing HFC-1225zc and hydrogen fluoride and a first bottoms comprising HFC-236fa; b) subjecting said first distillate to a second distillation step from which a composition enriched in either (i) hydrogen fluoride or (ii) HFC-1225zc is removed as a second distillate composition with a second bottoms composition being enriched in the other of said components (i) or (ii); and c) subjecting said second distillate composition to a third distillation step conducted at a different pressure than the second distillation step in which the component enriched in the second bottoms composition in (b) is removed in a third distillate composition with a third bottoms composition enriched in the same component that was enriched in the second distillate composition.

A further aspect relates to a process to produce HFC-1225zc comprising: a) feeding HFC-236fa to a reaction zone for dehydrofluorination to form a reaction product composition comprising HFC-1225zc, unreacted HFC-236fa and hydrogen fluoride; b) subjecting said reaction product composition to a first distillation step to form a first distillate composition comprising an azeotrope or near-azeotrope composition containing HFC-1225zc and hydrogen fluoride and a first bottoms composition comprising HFC-236fa; c) subjecting said first distillate composition to a second distillation step from which a composition enriched in either (i) hydrogen fluoride or (ii) HFC-1225zc is removed as a second distillate composition with a second bottoms composition being enriched in the other of said components (i) or (ii); and d) subjecting said second distillate composition to a third distillation step conducted at a different pressure than the second distillation step in which the component enriched in the second bottoms composition in (c) is removed in a third distillate composition with a third bottoms composition enriched in the same component that was enriched in the second distillate composition.

A further aspect relates to a process for the separation of HFC-236fa from a mixture comprising an azeotrope or near-azeotrope composition of HFC-236fa and HF, said process comprising: a) subjecting said mixture to a first distillation step in which a composition enriched in either (i) hydrogen fluoride or (ii) HFC-236fa is removed as a first distillate composition with a first bottoms composition being enriched in the other of said components (i) or (ii); and b) subjecting said first distillate composition to a second distillation step conducted at a different pressure in which the component enriched as first bottoms composition in (a) is removed in a second distillate composition with a second bottoms composition enriched in the same component which was enriched in the first distillate composition.

Another aspect provides a process for producing CF₃CH═CF₂ in the absence of dehydrofluorination catalyst. In particular, this aspect comprises pyrolyzing CF₃CH₂CF₃ to make CF₃CH═CF₂. Pyrolyzing accomplishes the thermal decomposition of the CF₃CH₂CF₃, at a temperature greater than about 700° C.

This selective formation of CF₃CH═CF₂ embodies several unexpected results. First, it is surprising that the heat input of the pyrolysis process does not cause the CF₃CH₂CF₃ reactant to fragment to C-1, e.g., methanes, and C-2, e.g., ethane and ethylene, compounds. Second, it is surprising that the CF₃CH═CF₂ product is stable under pyrolysis conditions and does not undergo further conversion to rearranged products or to products containing fewer hydrogen and/or fluorine atoms. Third, it is surprising that the pyrolysis to form CF₃CH═CF₂ takes place with high selectivity.

### BRIEF DESCRIPTION OF THE DRAWING(S)

FIG. 1 is a schematic flow diagram illustrating one embodiment for practicing a two-column azeotropic distillation process.

FIG. 2 is a schematic flow diagram illustrating one embodiment for practicing a process for production of HFC-1225zc.

### DETAILED DESCRIPTION OF THE INVENTION

One aspect relates to compositions containing 1,1,3,3,3-pentafluoropropene (HFC-1225zc, CF₃CH═CF₂, CAS reg. no. 690-27-7) which finds use as a co-monomer in some polymerization reactions to provide a site of specific chemical reactivity on the polymer backbone. HFC-1225zc may be prepared by methods known in the art such as U.S. Pat. No. 6,369,284, incorporated herein by reference.

Anhydrous hydrogen fluoride (HF) has CAS reg. no. 7664-39-3 and is commercially available.

Also useful in the processes disclosed herein is 1,1,1,3,3,3-hexafluoropropane (HFC-236fa, CAS reg. no. 690-39-1). HFC-236fa may be prepared by methods known in the art and is commercially available.

In considering a process for the dehydrofluorination of HFC-236fa to HFC-1225zc and HF and the isolation of HFC-1225zc from such a process, it has been discovered surprisingly that the hydrofluoroolefin HFC-1225zc forms an azeotrope with HF.

One aspect provides a composition, which comprises HFC-1225zc and an effective amount of hydrogen fluoride (HF) to form an azeotrope composition. By effective amount is meant an amount, which, when combined with HFC-1225zc, results in the formation of an azeotrope or near-azeotrope mixture. As recognized in the art, an azeotrope or a near-azeotrope composition is an admixture of two or more different components which, when in liquid form under a given pressure, will boil at a substantially constant temperature, which temperature may be higher or lower than the boiling temperatures of the individual components, and which will provide a vapor composition essentially identical to the liquid composition undergoing boiling.

For the purpose of this discussion, near-azeotrope composition (also commonly referred to as an “azeotrope-like composition”) means a composition that behaves like an azeotrope (i.e., has constant boiling characteristics or a tendency not to fractionate upon boiling or evaporation). Thus, the composition of the vapor formed during boiling or evaporation is the same as or substantially the same as the original liquid composition. Hence, during boiling or evaporation, the liquid composition, if it changes at all, changes only to a minimal or negligible extent. This is to be contrasted with non-azeotrope compositions in which during boiling or evaporation, the liquid composition changes to a substantial degree.

Additionally, near-azeotrope compositions exhibit dew point pressure and bubble point pressure with virtually no pressure differential. That is to say that the difference in the dew point pressure and bubble point pressure at a given temperature will be a small value. It may be stated that compositions with a difference in dew point pressure and bubble point pressure of less than or equal to 3 percent (based upon the bubble point pressure) may be considered to be a near-azeotrope.

Accordingly, the essential features of an azeotrope or a near-azeotrope composition are that at a given pressure, the boiling point of the liquid composition is fixed and that the composition of the vapor above the boiling composition is essentially that of the boiling liquid composition (i.e., no fractionation of the components of the liquid composition takes place). It is also recognized in the art that both the boiling point and the weight percentages of each component of the azeotrope composition may change when the azeotrope or near-azeotrope liquid composition is subjected to boiling at different pressures. Thus, an azeotrope or a near-azeotrope composition may be defined in terms of the unique relationship that exists among the components or in terms of the compositional ranges of the components or in terms of exact weight percentages of each component of the composition characterized by a fixed boiling point at a specified pressure. It is also recognized in the art that various azeotrope compositions (including their boiling points at particular pressures) may be calculated (see, e.g., W. Schotte Ind. Eng. Chem. Process Des. Dev. (1980) 19, 432-439). Experimental identification of azeotrope compositions involving the same components may be used to confirm the accuracy of such calculations and/or to modify the calculations at the same or other temperatures and pressures.

Compositions may be formed that comprise azeotrope combinations of hydrogen fluoride with HFC-1225zc. These include compositions comprising from about 29.6 mole percent to about 38.0 mole percent HF and from about 70.4 mole percent to about 62.0 mole percent HFC-1225zc (which forms an azeotrope boiling at a temperature from between about −20° C. and about 100° C. and at a pressure from between about 17.4 psi (120 kPa) and about 745 psi (5137 kPa).

Additionally, near-azeotrope compositions containing HF and HFC-1225zc may also be formed. Such near-azeotrope compositions comprise about 57.8 mole percent to about 82.0 mole percent HFC-1225zc and about 42.2 mole percent to about 18.0 mole percent HF at temperatures ranging from about −20° C. to about 100° C. and at pressures from about 17.4 psi (120 kPa) and about 745 psi (5137 kPa).

It should be understood that while an azeotrope or near-azeotrope composition may exist at a particular ratio of the components at given temperatures and pressures, the azeotrope composition may also exist in compositions containing other components. These additional components include the individual components of the azeotrope composition, said components being present as an excess above the amount being present as the azeotrope composition. For instance, the azeotrope of HFC-1225zc and HF may be present in a composition that has an excess of HFC-1225zc, meaning that the azeotrope composition is present and additional HFC-1225zc is also present.

Compositions may be formed that consist essentially of azeotrope combinations of hydrogen fluoride with HFC-1225zc. These include compositions consisting essentially of from about 29.6 mole percent to about 38.0 mole percent HF and from about 70.4 mole percent to about 62.0 mole percent HFC-1225zc (which forms an azeotrope boiling at a temperature from between about −20° C. and about 100° C. and at a pressure from between about 17.4 psi (120 kPa) and about 745 psi (5137 kPa).

Near azeotrope compositions may also be formed that consist essentially of about 57.8 mole percent to about 82.0 mole percent HFC-1225zc and about 42.2 mole percent to about 18.0 mole percent HF at temperatures ranging from about −20° C. to about 100° C. and at pressures from about 17.4 psi (120 kPa) and about 745 psi (5137 kPa).

At atmospheric pressure, the boiling points of hydrofluoric acid and HFC-1225zc are about 19.5° C. and −21° C., respectively. The relative volatility at 39.2 psi (270 kPa) and 0.3° C. of HF and HFC-1225zc was found to be nearly 1.0 as 30.9 mole percent HF and 69.1 mole percent HFC-1225zc was approached. The relative volatility at 183 psi (1262 kPa) and 50.1° C. was found to be nearly 1.0 as 34.5 mole percent HF and 65.5 mole percent HFC-1225zc was approached. These data indicate that the use of conventional distillation procedures will not result in the separation of a substantially pure compound because of the low value of relative volatility of the compounds.

To determine the relative volatility of HF with HFC-1225zc, the so-called PTx Method was used. In this procedure, the total absolute pressure in a cell of known volume is measured at a constant temperature for various known binary compositions. Use of the PTx Method is described in greater detail in “Phase Equilibrium in Process Design”, Wiley-Interscience Publisher, 1970, written by Harold R. Null, on pages 124 to 126, the entire disclosure of which is hereby incorporated by reference. Samples of the vapor and liquid, or vapor and each of the two liquid phases under those conditions where two liquid phases exist, were obtained and analyzed to verify their respective compositions.

These measurements can be reduced to equilibrium vapor and liquid compositions in the cell by an activity coefficient equation model, such as the Non-Random, Two-Liquid (NRTL) equation, to represent liquid phase non-idealities. Use of an activity coefficient equation, such as the NRTL equation, is described in greater detail in “The Properties of Gases and Liquids”, 4^{th} Edition, publisher McGraw Hill, written by Reid, Prausnitz and Poling, on pages 241 to 387; and in “Phase Equilibria in Chemical Engineering”, published by Butterworth Publishers, 1985, written by Stanley M. Walas, pages 165 to 244; the entire disclosure of each of the previously identified references are hereby incorporated by reference.

Without wishing to be bound by any theory or explanation, it is believed that the NRTL equation can sufficiently predict whether or not mixtures of HF and HFC-1225zc behave in an ideal manner, and can sufficiently predict the relative volatilities of the components in such mixtures. Thus, while HF has a good relative volatility compared to HFC-1225zc at low HFC-1225zc concentrations, the relative volatility becomes nearly 1.0 as 69.1 mole percent HFC-1225zc was approached at 0.3° C. This would make it impossible to separate HFC-1225zc from HF by conventional distillation from such a mixture. Where the relative volatility approaches 1.0 defines the system as forming a near-azeotrope or azeotrope composition.

It has been found that azeotropes of HFC-1225zc and HF are formed at a variety of temperatures and pressures. Azeotrope compositions may be formed between 17.4 psi (120 kPa) at a temperature of −20° C., and 745 psi (5137 kPa) at a temperature of 100° C., said compositions consisting essentially of HFC-1225zc and HF range from about 29.6 mole percent HF (and 70.4 mole percent HFC-1225zc) to about 38.0 mole percent HF (and 62.0 mole percent HFC-1225zc). An azeotrope of HF and HFC-1225zc has been found at 0.3° C. and 39.2 psi (270 kPa) consisting essentially of about 30.9 mole percent HF and about 69.1 mole percent HFC-1225zc. An azeotrope of HF and HFC-1225zc has also been found at 50.1° C. and 183 psi (1262 kPa) consisting essentially of about 34.5 mole percent HF and about 65.5 mole percent HFC-1225zc. Based upon the above findings, azeotrope compositions at other temperatures and pressures may be calculated. It has been calculated that an azeotrope composition of about 29.6 mole percent HF and about 70.4 mole percent HFC-1225zc can be formed at −20° C. and 17.4 psi (120 kPa) and an azeotrope composition of about 29.6 mole percent HF and about 70.4 mole percent HFC-1225zc can be formed at 100° C. and 745 psi (5137 kPa). Accordingly, one aspect provides an azeotrope composition consisting essentially of from about 29.6 mole percent to about 38.0 mole percent HF and from about 70.4 mole percent to about 62.0 mole percent HFC-1225zc, said composition having a boiling point of about −20° C. at 17.4 psi (120 kPa) to about 100° C. at 745 psi (5137 kPa).

It has also been found that azeotrope or near-azeotrope compositions may be formed between about 17.4 psi (120 kPa) to about 745 psi (5137 kPa) at temperatures ranging from about −20° C. to about 100° C., said compositions consisting essentially of about 57.8 mole percent to about 82.0 mole percent HFC-1225zc and about 42.2 mole percent to about 18.0 mole percent HF.

The HF/HFC-1225zc azeotrope and near-azeotrope compositions are useful in processes to produce HFC-1225zc and in processes to purify HFC-1225zc. In fact, the HF/HFC-1225zc azeotrope and near-azeotrope compositions may be useful in any process that creates a composition containing HFC-1225zc and HF.

Azeotropic distillation may be carried out to separate HFC-1225zc from HFC-236fa, which is the starting material for production of HFC-1225zc, by vapor phase dehydrofluorination. A two-column azeotropic distillation may then be carried out to separate the co-produced HF from the desired HFC-1225zc product. And another two-column azeotropic distillation may be carried out to separate HF from HFC-236fa. HF may be removed from the halogenated hydrocarbon components of the product mixture using, for example, standard aqueous solution scrubbing techniques. However, the production of substantial amounts of scrubbing discharge can create aqueous waste disposal concerns. Thus, there remains a need for processes utilizing HF from such product mixtures.

While the initial mixture treated in accordance with the processes disclosed herein can be obtained from a variety of sources, including by adding HFC-1225zc to HF-containing compositions, an advantageous use of the present processes resides in treating the effluent mixtures from the preparation of HFC-1225zc.

HFC-1225zc may be prepared by the vapor phase dehydrofluorination of HFC-236fa by processes known in the art, such as those described in U.S. Pat. No. 6,369,284, incorporated herein by reference.

Another aspect provides a process for the separation of HFC-1225zc from HFC-236fa comprising: a) forming a mixture of HFC-1225zc, HFC-236fa, and hydrogen fluoride; and b) subjecting said mixture to a distillation step forming a column distillate composition comprising an azeotrope or near-azeotrope composition of HF and HFC-1225zc essentially free of HFC-236fa.

As described herein, by “essentially free of HFC-236fa” is meant that the composition contains less than about 100 ppm (mole basis), preferably less than about 10 ppm and most preferably less than about 1 ppm, of HFC-236fa.

This azeotropic distillation takes advantage of the low boiling azeotrope composition formed by HFC-1225zc and HF. The azeotrope composition boils at a temperature lower than the boiling point of either pure component and lower than the boiling point of HFC-236fa as well.

As stated previously, the mixture of HFC-1225zc, HFC-236fa and HF may be formed by any practical means. Generally, the present process is particularly useful for the separation of HFC-1225zc from the reaction mixture produced by the dehydrofluorination of HFC-236fa. HF is a co-product formed in this dehydrofluorination reaction. The reaction mixture produced may then be treated by the instant process to remove HFC-236fa. The HFC-1225zc is taken overhead as the distillate from the distillation column as an azeotrope or near-azeotrope composition of HFC-1225zc with HF. The HFC-236fa is taken out of the bottom of the column as a bottoms composition and may contain some amount of HF, as well. The amount of HF in the HFC-236fa from the bottom of the distillation column may vary from about 37 mole percent to less than 1 part per million (ppm, mole basis) depending on the manner in which the dehydrofluorination reaction is conducted. In fact, if the dehydrofluorination reaction is conducted in a manner to provide 50 percent conversion of the HFC-236fa and the reaction mixture leaving the reaction zone is fed directly to the distillation step, the HFC-236fa leaving the bottom of the distillation process will contain about 36 mole percent HF.

In one embodiment, operating the present azeotropic distillation involves providing an excess of HFC-1225zc to the distillation column. If the proper amount of HFC-1225zc is fed to the column, then all the HF may be taken overhead as an azeotrope composition containing HFC-1225zc and HF. Thus, the HFC-236fa removed from the column bottoms will be essentially free of HF.

As described herein, by “essentially free of HF” is meant that the composition contains less than about 100 ppm (mole basis), preferably less than about 10 ppm and most preferably less than about 1 ppm, of HF.

In the distillation step, the distillate exiting the distillation column overhead comprising HF and HFC-1225zc may be condensed using, for example, standard reflux condensers. At least a portion of this condensed stream may be returned to the top of the column as reflux. The ratio of the condensed material, which is returned to the top of the distillation column as reflux, to the material removed as distillate is commonly referred to as the reflux ratio. The specific conditions which may be used for practicing the distillation step depend upon a number of parameters, such as the diameter of the distillation column, feed points, and the number of separation stages in the column, among others. The operating pressure of the distillation column may range from about 10 psi pressure to about 200 psi (1380 kPa), normally about 10 psi to about 50 psi. The distillation column is typically operated at a pressure of about 20 psi (138 kPa) with a bottoms temperature of about 8° C. and a tops temperature of about −16° C. Normally, increasing the reflux ratio results in increased distillate stream purity, but generally the reflux ratio ranges between 1/1 to 100/1. The temperature of the condenser, which is located adjacent to the top of the column, is normally sufficient to substantially fully condense the distillate that is exiting from the top of the column, or is that temperature required to achieve the desired reflux ratio by partial condensation.

The column distillate composition comprising an azeotrope or near-azeotrope composition of HF and HFC-1225zc, essentially free of HFC-236fa, must be treated to remove the HF and provide pure HFC-1225zc as product. This may be accomplished, for example, by neutralization or by a second distillation process, as described herein.

A further aspect provides a process for the separation of HFC-1225zc from a mixture comprising an azeotrope or near-azeotrope composition of HFC-1225zc and HF, said process comprising: a) subjecting said mixture to a first distillation step in which a composition enriched in either (i) hydrogen fluoride or (ii) HFC-1225zc is removed as a first distillate composition with a first bottoms composition being enriched in the other of said components (i) or (ii); and b) subjecting said first distillate composition to a second distillation step conducted at a different pressure than the first distillation step in which the component enriched as first bottoms composition in (a) is removed in a second distillate composition with a second bottoms composition enriched in the same component which was enriched in the first distillate composition.

The process as described above takes advantage of the change in azeotrope composition at different pressures to effectuate the separation of HFC-1225zc and HF. The first distillation step may be carried out at high pressure relative to the second distillation step. At higher pressures, the HF/HFC-1225zc azeotrope contains less HFC-1225zc. Thus, this high-pressure distillation step produces an excess of HFC-1225zc, which boiling at a higher temperature than the azeotrope will exit the column as the bottoms as pure HFC-1225zc. The first column distillate is then fed to a second distillation step operating at lower pressure. At the lower pressure, the HF/HFC-1225zc azeotrope shifts to lower concentrations of HF. Therefore, in this second distillation step, there exists an excess of HF. The excess HF, having a boiling point higher than the azeotrope, exits the second distillation column as the bottoms composition. The present process may be conducted in such as manner as to produce HFC-1225zc essentially free of HF. Additionally, the present preocess may be conducted in such a manner as to produce HF essentially free of HFC-1225zc.

Alternatvely, the first distillation step may be carried out at low pressure relative to the second distillation step. At lower pressures, the HF/HFC-1225zc azeotrope contains less HF. Thus, this low-pressure distillation step produces an excess of HF, which boiling at a higher temperature than the azeotrope will exit the column as the bottoms as pure HF. The first column distillate is then fed to a second distillation step operating at higher pressure. At the higher pressure, the HF/HFC-1225zc azeotrope shifts to lower concentrations of HFC-1225zc. Therefore, in this second distillation step, there exists an excess of HFC-1225zc. The excess HFC-1225zc, having a boiling point higher than the azeotrope, exits the second distillation column as the bottoms composition. The present process may be conducted in such as manner as to produce HFC-1225zc essentially free of HF. Additionally, the present process may be conducted in such a manner as to produce HF essentially free of HFC-1225zc.

As described herein, by “essentially free of HFC-1225zc” is meant that the composition contains less than about 100 ppm (mole basis), preferably less than about 10 ppm and most preferably less than about 1 ppm, of HFC-1225zc.

The endothermic dehydrofluorination reaction of HFC-236fa to produce HFC-1225zc may be accomplished, for example, in a tubular reactor with catalyst in the tubes and with a heating medium on the shellside of the reactor. Alternatively, a heat carrier may be used to permit adiabatic operation. Either pure HFC-236fa or pure HFC-1225zc, both being produced by the distillation processes described herein, may be recycled back to the reactor to serve as heat carrier. HFC-236fa would be a preferred heat carrier, as introduction of HFC-1225zc to the dehydrofluorination reactor will result in a reduction in single-pass conversion of HFC-236fa.

In both the first and second distillation steps, the distillate exiting the distillation column overhead comprising HF and HFC-1225zc may be condensed using, for example, standard reflux condensers. At least a portion of this condensed stream may be returned to the top of the column as reflux. The ratio of the condensed material, which is returned to the top of the distillation column as reflux, to the material removed as distillate is commonly referred to as the reflux ratio. The specific conditions which may be used for practicing the distillation step depend upon a number of parameters, such as the diameter of the distillation column, feed points, and the number of separation stages in the column, among others. The operating pressure of the high pressure (whether the high pressure distillation column is the first or second column) distillation column may range from about 100 psi (689 kPa) pressure to about 400 psi (2760 kPa), normally about 200 psi (1380 kPa) to about 350 psi (2410 kPa). The high pressure distillation column is typically operated at a pressure of about 315 psi (2170 kPa) with a bottoms temperature of about 80° C. and a tops temperature of about 72° C. Normally, increasing the reflux ratio results in increased distillate stream purity, but generally the reflux ratio ranges between 0.1/1 to 100/1. The temperature of the condenser, which is located adjacent to the top of the column, is normally sufficient to substantially fully condense the distillate that is exiting from the top of the column, or is that temperature required to achieve the desired reflux ratio by partial condensation.

The operating pressure of the low pressure (whether the low pressure distillation column is the first or second distillation column) distillation column may range from about 5 psi (34 kPa) pressure to about 50 psi (345 kPa), normally about 10 psi (69 kPa) to about 30 psi (207 kPa). The low pressure distillation column is typically operated at a pressure of about 20 psi (138 kPa) with a bottoms temperature of about 31° C. and a tops temperature of about −17° C. Normally, increasing the reflux ratio results in increased distillate stream purity, but generally the reflux ratio ranges between 0.1/1 to 50/1. The temperature of the condenser, which is located adjacent to the top of the column, is normally sufficient to substantially fully condense the distillate that is exiting from the top of the column, or is that temperature required to achieve the desired reflux ratio by partial condensation.

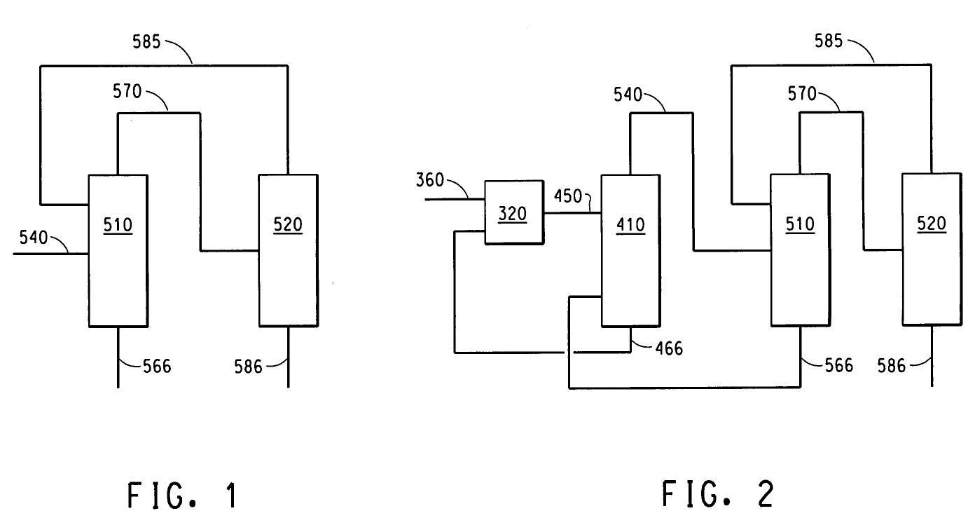

FIG. 1 is illustrative of one embodiment for practicing the present two-column distillation process for the separation of HFC-1225zc and HF. Referring to FIG. 1, a feed mixture derived from a prior azeotropic distillation comprising HF and HFC-1225zc, wherein the molar ratio of HF:HFC-1225zc is about 0.44:1 (or lower), is passed through line (540) to a multiple stage distillation column (510), operating at a temperature of about 72° C. and a pressure of about 315 psi (2170 kPa). The bottoms of the distillation column (510), containing essentially pure HFC-1225zc at a temperature of about 80° C. and a pressure of about 317 psi (2190 kPa) is removed from the bottom of column (510) through line (566). The distillate from column (510), containing the HF/HFC-1225zc azeotrope (HF:HFC-1225zc molar ratio is about 0.54:1) at a temperature of about 72° C. and a pressure of about 315 psi (2170 kPa) is removed from the top of column (510) and sent through line (570) to a multiple stage distillation column (520). The distillate from column (520), containing the HF/HFC-1225zc azeotrope (molar ratio is about 0.44:1) at a temperature of about −17° C. and a pressure of about 20 psi (138 kPa), is removed from column (520) through line (585) and is recycled back to column (510). The bottoms of column (520) containing essentially pure HF at a temperature of about 31° C. and a pressure of about 22 psi (152 kPa) is removed through line (586).

U.S. Pat. No. 6,388,147, incorporated herein by reference, discloses azeotrope and near-azeotrope compositions consisting essentially of HFC-236fa and HF ranging from about 41 mole percent to about 63 mole percent HFC-236fa and from about 59 mole percent to about 37 mole percent HF. The existence of this azeotrope allows the separation of HFC-236fa from HF to be accomplished in a similar manner to the separation of HFC-1225zc from HF, that being a two-column azeotropic distillation.

A further aspect provides a process for the separation of HFC-236fa from a mixture comprising an azeotrope or near-azeotrope composition of HFC-236fa and HF, said process comprising: a) subjecting said mixture to a first distillation step in which a composition enriched in either (i) hydrogen fluoride or (ii) HFC-236fa is removed as a first distillate composition with a first bottoms composition being enriched in the other of said components (i) or (ii); and b) subjecting said first distillate composition to a second distillation step conducted at a different pressure in which the component enriched as first bottoms composition in (a) is removed in a second distillate composition with a second bottoms composition enriched in the same component which was enriched in the first distillate composition.

Similar to the previously described two-column azeotropic distillation, for both the first and second distillation steps, the distillate exiting the distillation column overhead comprising HF and HFC-236fa may be condensed using, for example, standard reflux condensers. At least a portion of this condensed stream may be returned to the top of the column as reflux. The ratio of the condensed material, which is returned to the top of the distillation column as reflux, to the material removed as distillate is commonly referred to as the reflux ratio. The specific conditions which may be used for practicing the distillation step depend upon a number of parameters, such as the diameter of the distillation column, feed points, and the number of separation stages in the column, among others. The operating pressure of the low pressure (whether the low pressure distillation column is the first or second column) distillation column may range from about 5 psi (34 kPa) pressure to about 50 psi (345 kPa), normally about 10 psi (70 kPa) to about 30 psi (209 kPa). The low pressure distillation column is typically operated at a pressure of about 20 psi (138 kPa) with a bottoms temperature of about 8° C. and a tops temperature of about 0° C. Normally, increasing the reflux ratio results in increased distillate stream purity, but generally the reflux ratio ranges between 0.1/1 to 50/1. The temperature of the condenser, which is located adjacent to the top of the column, is normally sufficient to substantially fully condense the distillate that is exiting from the top of the column, or is that temperature required to achieve the desired reflux ratio by partial condensation.

The operating pressure of the high pressure (whether the high pressure distillation column is the first or second column) distillation column may range from about 100 psi (690 kPa) pressure to about 400 psi (2760 kPa), normally about 200 psi (1380 kPa) to about 350 psi (2410 kPa). The high pressure distillation column is typically operated at a pressure of about 315 psi (2170 kPa) with a bottoms temperature of about 133° C. and a tops temperature of about 91° C. Normally, increasing the reflux ratio results in increased distillate stream purity, but generally the reflux ratio ranges between 0.1/1 to 50/1. The temperature of the condenser, which is located adjacent to the top of the column, is normally sufficient to substantially fully condense the distillate that is exiting from the top of the column, or is that temperature required to achieve the desired reflux ratio by partial condensation.

FIG. 1 is also illustrative of one embodiment for practicing the present two-column distillation process for separation of HFC-236fa and HF. Referring to FIG. 1, a feed mixture derived from a prior azeotropic distillation comprising HFC-236fa and HF, wherein the molar ratio of HF:HFC-236fa is about 1.2:1 (or lower), is passed through line (540) to a multiple stage distillation column (510), operating at a temperature of about 0° C. and a pressure of about 20 psi (138 kPa). The bottoms of the distillation column (510), containing essentially pure HFC-236fa at a temperature of about 8° C. and a pressure of about 22 psi (152 kPa) is removed from the bottom of column (510) through line (566). The distillate from column (510), containing the HF/HFC-236fa azeotrope (HF:HFC-236fa molar ratio is about 1.2:1) at a temperature of about 0° C. and a pressure of about 20 psi (138 kPa) is removed from the top of column (510) and sent through line (570) to a multiple stage distillation column (520). The distillate from column (520), containing the HF/HFC-236fa azeotrope (molar ratio is about 0.85:1) at a temperature of about 91° C. and a pressure of about 315 psi (2170 kPa), is removed from column (520) through line (585) and is recycled back to column (510). The bottoms of column (520) containing essentially pure HF at a temperature of about 133° C. and a pressure of about 317 psi (2190 kPa) is removed through line (586).

A further aspect provides a process for the purification of HFC-1225zc from a mixture of HFC-1225zc, HFC-236fa, and HF, said process comprising: a) subjecting said mixture to a first distillation step to form a first distillate comprising an azeotrope or near-azeotrope composition containing HFC-1225zc and HF and a first bottoms comprising HFC-236fa; b) subjecting said first distillate to a second distillation step from which a composition enriched in either (i) hydrogen fluoride or (ii) HFC-1225zc is removed as a second distillate composition with a second bottoms composition being enriched in the other of said components (i) or (ii); and c) subjecting said second distillate composition to a third distillation step conducted at a different pressure than the second distillation step in which the component enriched in the second bottoms composition in (b) is removed in a third distillate composition with a third bottoms composition enriched in the same component that was enriched in the second distillate composition.

A further aspect provides a process to produce HFC-1225zc comprising: a) feeding HFC-236fa to a reaction zone for dehydrofluorination to form a reaction product composition comprising HFC-1225zc, unreacted HFC-236fa and hydrogen fluoride; b) subjecting said reaction product composition to a first distillation step to form a first distillate composition comprising an azeotrope or near-azeotrope composition containing HFC-1225zc and HF and a first bottoms composition comprising HFC-236fa; c) subjecting said first distillate composition to a second distillation step from which a composition enriched in either (i) hydrogen fluoride or (ii) HFC-1225zc is removed as a second distillate composition with a second bottoms composition being enriched in the other of said components (i) or (ii); and d) subjecting said second distillate composition to a third distillation step conducted at a different pressure than the second distillation step in which the component enriched in the second bottoms composition in (c) is removed in a third distillate composition with a third bottoms composition enriched in the same component that was enriched in the second distillate composition. Optionally, the process may further comprise recycling at least some portion of said first bottoms composition (HFC-236fa) to said reaction zone. Optionally, the process may further comprise recycling at least some portion of said second bottoms composition or third bottoms composition (whichever happens to be HFC-1225zc) to said reaction zone. Optionally, the process may further comprise recycling at least some portion of said second bottoms composition or third bottoms composition (whichever happens to be HFC-1225zc) to said first distillation step. Optionally, the process may further comprise recovering at least some portion of said second bottoms composition or said third bottoms composition as HFC-1225zc essentially free of HFC-236fa and HF.

As described herein, by “essentially free of HFC236fa and HF” is meant that the composition contains less than about 100 ppm (mole basis), preferably less than about 10 ppm and most preferably less than about 1 ppm, of each of HFC-236fa and HF.

The reaction zone for the dehydrofluorination may comprise a flow reactor preferably containing a fixed bed of dehydrofluorination catalyst. The process equipment for all the processes disclosed herein and the associated feed lines, effluent lines and associated units may be constructed of materials resistant to hydrogen fluoride. Typical materials of construction, well-known to the art, include stainless steels, in particular of the austenitic type, and the well-known high nickel alloys such as Monel® nickel-copper alloys, Hastelloy® nickel based alloys and Inconel® nickel-chromium alloys.

FIG. 2 is illustrative of one embodiment for practicing the present process for production of HFC-1225zc. HFC-236fa is fed through line (360) to reactor (320). The reactor effluent mixture comprising HF, HFC-236fa and HFC-1225zc, exits the reactor through line (450) and is fed to a multiple stage distillation column (410). The bottoms of distillation column (410), containing essentially pure HFC-236fa is removed from the bottom of column (410) through line (466) and may be recycled back to the reactor (320) as a heat carrier. The distillate from column (410), containing the HF/HFC-1225zc azeotrope is removed from the top of column (410) and is sent through line (540) to a second multiple stage distillation column (510). The bottoms from column (510), which in this instance is essentially pure HFC-1225zc, is removed from column (510) through line (566) and may be recycled back to the first distillation column (410). The distillate from column (510), containing the HF/HFC-1225zc azeotrope, is fed through line (570) to a third multiple stage distillation column (520). The distillate from column (520) comprising HF/HFC-1225zc is removed through line (585) and may be recycled to the second distillation column (510). The bottoms composition from column (520) is essentially pure HF and is removed from column (520) through line (586). The essentially pure HF product from this process may be used in any manner appropriate such as feeding to a fluorination reactor for production of a fluorochemical compound, or may be neutralized for disposal.

While not illustrated in the figures, it is understood that certain pieces of process equipment may be used in the processes described herein, for optimization. For instance, pumps, heaters or coolers may be used where appropriate. As an example, it is desirable to have the feed to a distillation column at the same temperature as the point in the column to which it is fed. Therefore, heating or cooling of the process stream may be necessary to match the temperature.

Another embodiment provides a process of producing CF₃CH═CF₂ by pyrolysis of CF₃CH₂CF₃. The process may be written as:
CF₃CH₂CF₃+Δ→CF₃CH═CF₂+HF
where Δ represents heat and HF is hydrogen fluoride.

Pyrolysis, as the term is used herein, means chemical change produced by heating in the absence of catalyst. Pyrolysis reactors generally comprise three zones: a) a preheat zone, in which reactants are brought close to the reaction temperature; b) a reaction zone, in which reactants reach reaction temperature and are at least partially pyrolyzed, and products and any byproducts form; c) a quench zone, in which the stream exiting the reaction zone is cooled to stop the pyrolysis reaction. Laboratory-scale reactors have a reaction zone, but the preheating and quenching zones may be omitted.

In this embodiment, the reactor may be of any shape consistent with the process but is preferably a cylindrical tube, either straight or coiled. Although not critical, such reactors typically have an inner diameter of from about 1.3 to about 5.1 cm (about 0.5 to about 2 inches). Heat is applied to the outside of the tube, the chemical reaction taking place on the inside of the tube. The reactor and its associated feed lines, effluent lines and associated units should be constructed, at least as regards the surfaces exposed to the reaction reactants and products, of materials resistant to hydrogen fluoride. Typical materials of construction, well-known to the fluorination art, include stainless steels, in particular of the austenitic type, the well-known high nickel alloys, such as Monel® nickel-copper alloys, Hastelloy-based alloys and Inconel® nickel-chromium alloys and copper clad steel. Where the reactor is exposed to high temperature, the reactor may be constructed of more than one material. For example, the outer surface layer of the reactor should be chosen for ability to maintain structural integrity and resist corrosion at the pyrolysis temperature, the inner surface layer of the reactor should be chosen of materials resistant to attack by, that is, inert to, the reactant and products. In the case of the present process, the product hydrogen fluoride is corrosive to certain materials. In other words, the reactor may be constructed of an outer material chosen for physical strength at high temperature and an inner material chosen for resistance to corrosion by the reactants and products under the temperature of the pyrolysis.

For the process of this embodiment, it is preferred that the reactor inner surface layer be made of high nickel alloy, that is an alloy containing at least about 50 wt % nickel, preferably a nickel alloy having at least about 75 wt % nickel, more preferably a nickel alloy having less than about 8 wt % chromium, still more preferably a nickel alloy having at least about 98 wt % nickel, and most preferably substantially pure nickel, such as the commercial grade known as Nickel 200. More preferable than nickel or its alloys as the material for the inner surface layer of the reactor is gold. The thickness of the inner surface layer does not substantially affect the pyrolysis and is not critical so long as the integrity of the inner surface layer is intact. The thickness of the inner surface layer is typically from about 10 to about 100 mils (0.25 to 2.5 mm). The thickness of the inner surface layer can be determined by the method of fabrication, the cost of materials, and the desired reactor life.

The reactor outer surface layer is resistant to oxidation or other corrosion and maintains sufficient strength at the reaction temperatures to keep the reaction vessel from failing of distorting. This layer is preferably Inconel® alloy, more preferably Inconel® 600.

The present pyrolysis of CF₃CH₂CF₃ to CF₂═CHCF₃ and HF is carried out in the absence of catalyst in a substantially empty reactor. By absence of catalyst is meant that no material or treatment is added to the pyrolysis reactor that increases the reaction rate by reducing the activation energy of the pyrolysis process. It is understood that although surfaces that are unavoidably present in any containment vessel, such as a pyrolysis reactor, may have incidental catalytic or anticatalytic effects on the pyrolysis process, the effect makes an insignificant contribution, if any, to the pyrolysis rate. More specifically, absence of catalyst means absence of conventional catalysts having high surface area in a particulate, pellet, fibrous or supported form that are useful in promoting the elimination of hydrogen fluoride from a hydrofluorocarbon (i.e., dehydrofluorination). Exemplary dehydrofluorination catalysts include: chromium oxide, optionally containing other metals, metal oxides or metal halides; chromium fluoride, unsupported or supported; and activated carbon, optionally containing other metals, metal oxides or metal halides.

Substantially empty reactors useful for carrying out the present process are tubes comprising the aforementioned materials of construction. Substantially empty reactors include those wherein the flow of gases through the reactor is partially obstructed to cause back-mixing, i.e. turbulence, and thereby promote mixing of gases and good heat transfer. This partial obstruction can be conveniently obtained by placing packing within the interior of the reactor, filling its cross-section or by using perforated baffles. The reactor packing can be particulate or fibrillar, preferably in cartridge disposition for ease of insertion and removal, has an open structure like that of Raschig Rings or other packings with a high free volume, to avoid the accumulation of coke and to minimize pressure drop, and permits the free flow of gas. Preferably the exterior surface of such reactor packing comprises materials identical to those of the reactor inner surface layer; materials that do not catalyze dehydrofluorination of hydrofluorocarbons and are resistant to hydrogen fluoride. The free volume is the volume of the reaction zone minus the volume of the material that makes up the reactor packing. The free volume is at least about 80%, preferably at least about 90%, and more preferably about 95%.

The pyrolysis which accomplishes the conversion of CF₃CH₂CF₃ to CF₂═CHCF₃ is suitably conducted at a temperature of at least about 700° C., preferably at least about 750° C., and more preferably at least about 800° C. The maximum temperature is no greater than about 1,000° C., preferably no greater than about 950° C., and more preferably no greater than about 900° C. The pyrolysis temperature is the temperature of the gases inside at about the mid-point of the reaction zone.

The residence time of gases in the reaction zone is typically from about 0.5 to about 60 seconds, more preferably from about 2 seconds to about 20 seconds at temperatures of from about 700 to about 900° C. and atmospheric pressure. Residence time is determined from the net volume of the reaction zone and the volumetric feed rate of the gaseous feed to the reactor at a given reaction temperature and pressure, and refers to the average amount of time a volume of gas remains in the reaction zone.

The pyrolysis is preferably carried out to a conversion of the CF₃CH₂CF₃ at least about 25%, more preferably to at least about 35%, and most preferably to at least about 45%. By conversion is meant the portion of the reactant that is consumed during a single pass through the reactor. Pyrolysis is preferably carried out to a yield of CF₃CH═CF₂ of at least about 50%, more preferably at least about 60%, and most preferably at least about 75%. By yield is meant the moles of CF₃CH═CF₂ produced per mole of CF₃CH₂CF₃ consumed.

The reaction is preferably conducted at subatmospheric, or atmospheric total pressure. That is, the reactants plus other ingredients are at subatmospheric pressure or atmospheric pressure. (If inert gases are present as other ingredients, as discussed below, the sum of the partial pressures of the reactants plus such ingredients is subatmospheric or atmospheric). Near atmospheric total pressure is more preferred. The reaction can be beneficially run under reduced total pressure (i.e., total pressure less than one atmosphere).

The reaction according to this embodiment can be conducted in the presence of one or more unreactive diluent gases, that is diluent gases that do not react under the pyrolysis conditions. Such unreactive diluent gases include the inert gases nitrogen, argon, and helium. Fluorocarbons that are stable under the pyrolysis conditions, for example, trifluoromethane and perfluorocarbons, may also be used as unreactive diluent gases. It has been found that inert gases can be used to increase the conversion of CF₃CH₂CF₃ to CF₃CH═CF₂. Of note are processes where the mole ratio of inert gas to CF₃CH₂CF₃ fed to the pyrolysis reactor is from about 5:1 to 1:1. Nitrogen is a preferred inert gas because of its comparatively low cost.

The present process produces a 1:1 molar mixture of HF and CF₃CH═CF₂ in the reactor exit stream. The reactor exit stream can also contain unconverted reactant, CF₃CH₂CF₃. The components of the reactor exit stream can be separated by conventional means, such as distillation. Hydrogen fluoride and CF₃CH═CF₂ form a homogenous low-boiling azeotrope containing about 60 mole percent CF₃CH═CF₂. The present process reactor exit stream can be distilled and the low-boiling HF and CF₃CH═CF₂ azeotrope taken off as a distillation column overhead stream, leaving substantially pure CF₃CH₂CF₃ as a distillation column bottom stream. Recovered CF₃CH₂CF₃ reactant may be recycled to the reactor. CF₃CH═CF₂ can be separated from its azeotrope with HF by conventional procedures, such as pressure swing distillation or by neutralization of the HF with caustic.

Without further elaboration, it is believed that one skilled in the art can, using the description herein, utilize the disclosed compositions and processes to their fullest extent. The following exemplary embodiments are, therefore, to be construed as merely illustrative, and do not constrain the remainder of the disclosure in any way whatsoever.

### EXAMPLES

### Example 1

### Phase Studies of Mixtures of HF and HFC-1225zc

A phase study was performed for a composition consisting essentially of HFC-1225zc and HF, wherein the composition was varied and the vapor pressures were measured at both 0.3° C. and 50.1° C. Based upon the data from the phase studies, azeotrope compositions at other temperature and pressures have been calculated.

Table 1 provides a compilation of experimental and calculated azeotrope compositions for HF and HFC-1225zc at specified temperatures and pressures.

TABLE 1

| Temperature, | Pressure, | | Mole % |
|---|---|---|---|
| ° C. | psi (kPa) | Mole % HF | HFC-1225zc |
| −20 | 17.4 (120) | 29.6 | 70.4 |
| −17 | 19.7 (136) | 29.9 | 70.1 |
| −3 | 34.7 (239) | 31.0 | 69.0 |
| 0 | 38.8 (267) | 30.9 | 69.1 |
| 0.3 | 39.2 (270) | 30.9 | 69.1 |
| 3.9 | 44.7 (308) | 31.0 | 69.0 |
| 20 | 76.7 (529) | 32.8 | 67.2 |
| 40 | 139 (958) | 34.0 | 66.0 |
| 50.1 | 183 (1262) | 34.5 | 65.5 |
| 60 | 236 (1627) | 34.9 | 65.1 |
| 65 | 268 (1848) | 35.1 | 64.9 |
| 70 | 304 (2096) | 35.3 | 64.7 |
| 71.5 | 314.7 (2170) | 35.4 | 64.6 |
| 75 | 344 (2372) | 35.6 | 64.4 |
| 80 | 389 (2682) | 35.3 | 64.7 |
| 85 | 441 (3041) | 36.0 | 64.0 |
| 90 | 524 (3613) | 36.9 | 63.1 |
| 95 | 629 (4337) | 37.4 | 62.6 |
| 100 | 745 (5137) | 38.0 | 62.0 |

### Example 2

### Dew Point and Bubble Point Vapor Pressures

The dew point and bubble point vapor pressures for compositions disclosed herein were calculated from measured and calculated thermodynamic properties. The near-azeotrope range is indicated by the minimum and maximum concentration of HFC-1225zc (mole percent, mol %) for which the difference in dew point and bubble point pressures is less than or equal to 3% (based upon bubble point pressure). The results are summarized in Table 2.

TABLE 2

| | Azeotrope | Near azeotrope compositions, | |
|---|---|---|---|
| Temperature, | composition, | mol % HFC-1225zc | |
| ° C. | mol % HFC-1225zc | Minimum | Maximum |
| −20 | 70.4 | 63.4 | 82.0 |
| 60 | 65.1 | 60.2 | 83.2 |
| 100 | 62.0 | 57.8 | 72.0 |

### Example 3

### Azeotropic Distillation for Separation of HFC-1225zc from HFC-236fa

A mixture of HF, HFC-1225zc, and HFC-236fa is fed to a distillation column for the purpose of purification of the HFC-1225zc. The data in Table 3 were obtained by calculation using measured and calculated thermodynamic properties.

TABLE 3

| Component or | | Column overhead | |
|---|---|---|---|
| variable | Column feed | (distillate) | Column bottoms |
| HFC-236fa, | 33.4 | 0.1 ppm | 63.3 |
| mol % | | | |
| HFC-1225zc, | 33.3 | 70.1 | 0.30 |
| mol % | | | |
| HF, mol % | 33.3 | 29.9 | 36.4 |
| Temp, ° C. | — | −17.1 | 1.9 |
| Pressure, psi | — | 19.7 (136) | 21.7 (150) |
| (kPa) | | | |

### Example 4

### Azeotropic Distillation for Separation of HFC-1225zc from HFC-236fa

A mixture of HF, HFC-1225zc, and HFC-236fa is fed to a distillation column for the purpose of purification of the HFC-1225zc. The data in Table 4 were obtained by calculation using measured and calculated thermodynamic properties.

TABLE 4

| Component or | | Column overhead | |
|---|---|---|---|
| variable | Column feed | (distillate) | Column bottoms |
| HFC-236fa, | 23.0 | 10 ppm | 99.99 |
| mol % | | | |
| HFC-1225zc, | 54.0 | 70.1 | 79 ppm |
| mol % | | | |
| HF, mol % | 23.0 | 29.9 | — |
| Temp, ° C. | — | −16.1 | 8.3 |
| Pressure, psi | — | 19.7 (136) | 21.7 (150) |
| (kPa) | | | |

### Example 5

### Azeotropic Distillation for Separation of HFC-1225zc from HFC-236fa

A mixture of HF, HFC-1225zc, and HFC-236fa is fed to a distillation column for the purpose of purification of the HFC-1225zc. The data in Table 5 were obtained by calculation using measured and calculated thermodynamic properties.

TABLE 5

| Component or | | Column overhead | |
|---|---|---|---|
| variable | Column feed | (distillate) | Column bottoms |
| HFC-236fa, | 27.3 | 10 ppm | 99.99 |
| mol % | | | |
| HFC-1225zc, | 63.6 | 87.5 | 117 ppm |
| mol % | | | |
| HF, mol % | 9.1 | 12.5 | — |
| Temp, ° C. | — | −15.6 | 8.3 |
| Pressure, psi | — | 19.7 (136) | 21.7 (150) |
| (kPa) | | | |

### Example 6

### Azeotropic Distillation for Separation of HFC-1225zc from HFC-236fa

A mixture of HF, HFC-1225zc, and HFC-236fa is fed to a distillation column for the purpose of purification of the HFC-1225zc. The data in Table 6 were obtained by calculation using measured and calculated thermodynamic properties.

TABLE 6

| Component or | | Column overhead | |
|---|---|---|---|
| variable | Column feed | (distillate) | Column bottoms |
| HFC-236fa, | 17.6 | 10 ppm | 99.99 |
| mol % | | | |
| HFC-1225zc, | 76.5 | 92.9 | 78 ppm |
| mol % | | | |
| HF, mol % | 5.9 | 7.1 | — |
| Temp, ° C. | — | −15.4 | 8.3 |
| Pressure, psi | — | 19.7 (13.6) | 21.7 (150) |
| (kPa) | | | |

### Example 7

### Two-Column Azeotropic Distillation for Separation of HFC-1225zc from HF

A mixture of HF and HFC-1225zc is fed to a distillation process for the purpose of purification of the HFC-1225zc. The data in Table 7 were obtained by calculation using measured and calculated thermodynamic properties. The numbers at the top of the columns refer to FIG. 1.

TABLE 7

| | | 570 | 566 | 585 | |
|---|---|---|---|---|---|
| | 540 | Column | HFC- | Column | 586 |
| Compound | Feed | (510) | 1225ye | (520) | HF |
| or variable | Mixture | distillate | product | distillate | product |
| HF, mol % | 30.4 | 35.0 | — | 30.5 | 100 |
| HFC- | 69.6 | 65.0 | 100 | 69.5 | — |
| 1225zc, mol % | | | | | |
| Temp., | — | 71.5 | 80.1 | −17.1 | 30.6 |
| ° C. | | | | | |
| Pres., | — | 314.7 | 316.7 | 19.7 | 21.7 |
| psi (kPa) | | (2170) | (2184) | (136) | (150) |

### Example 8

### Two-Column Azeotropic Distillation for Separation of HFC-1225zc from HF

A mixture of HF and HFC-1225zc is fed to a distillation process for the purpose of purification of the HFC-1225zc. The data in Table 8 were obtained by calculation using measured and calculated thermodynamic properties. The numbers at the top of the columns refer to FIG. 1.

TABLE 8

| | | 570 | 566 | 585 | |
|---|---|---|---|---|---|
| | 540 | Column | HFC- | Column | 586 |
| Compound | Feed | (510) | 1225ye | (520) | HF |
| or variable | Mixture | distillate | product | distillate | product |
| HF, mol % | 30.4 | 24.1 | — | 30.5 | 100 |
| HFC- | 69.6 | 75.9 | 100 | 69.5 | — |
| 1225zc, mol % | | | | | |
| Temp., | — | 71.5 | 80.1 | −17.1 | 30.6 |
| ° C. | | | | | |
| Pres., | — | 314.7 | 316.7 | 19.7 | 21.7 |
| psi (kPa) | | (2170) | (2184) | (136) | (150) |

### Example 9

### Two-Column Azeotropic Distillation for Separation of HFC-1225zc from HF

A mixture of HF and HFC-1225zc is fed to a distillation process for the purpose of purification of the HFC-1225zc. The data in Table 9 were obtained by calculation using measured and calculated thermodynamic properties. The numbers at the top of the columns refer to FIG. 1.

TABLE 9

| | | 570 | 566 | 585 | |
|---|---|---|---|---|---|
| | 540 | Column | HFC- | Column | 586 |
| Compound | Feed | (510) | 1225ye | (520) | HF |
| or variable | Mixture | distillate | product | distillate | product |
| HF, mol % | 19.0 | 35.0 | — | 30.5 | 100 |
| HFC- | 81.0 | 65.0 | 100 | 69.5 | — |
| 1225zc, mol % | | | | | |
| Temp., | — | 71.5 | 80.1 | −17.1 | 30.6 |
| ° C. | | | | | |
| Pres., | — | 314.7 | 316.7 | 19.7 | 21.7 |
| psi (kPa) | | (2170) | (2184) | (136) | (150) |

### Example 10

### Two-Column Azeotropic Distillation for Separation of HFC-236fa from HF

A mixture of HF and HFC-236fa is fed to a distillation process for the purpose of purification of the HFC-236fa. The data in Table 10 were obtained by calculation using measured and calculated thermodynamic properties. The numbers at the top of the columns refer to FIG. 1.

TABLE 10

| | | 570 | 566 | 585 | |
|---|---|---|---|---|---|
| | 540 | Column | HFC- | Column | 586 |
| Compound | Feed | (510) | 236fa | (520) | HF |
| or variable | Mixture | distillate | product | distillate | product |
| HF, mol % | 57.1 | 54.0 | — | 46.0 | 100 |
| HFC-236fa, | 42.9 | 46.0 | 100 | 54.0 | — |
| mol % | | | | | |
| Temp., | — | 0 | 8.3 | 90.5 | 132.9 |
| ° C. | | | | | |
| Pres., | — | 19.7 | 21.7 | 314.7 | 316.7 |
| psi (kPa) | | (136) | (150) | (2170) | (2184) |

### Examples 11-14

Examples 11-14 use one of three reactors:

Reactor A: Inconel® 600 tube (this alloy is about 76 wt % nickel), 18 in (45.7 cm) long×1.0 in (2.5 cm) outer diameter×0.84 in (2.1 cm) inner diameter. Tube wall thickness is 0.16 in (0.41 cm). The preheat zone is 7 in (17.8 cm) long. The reaction zone is 2 in (5.1 cm) long. The quench zone is 7 in (17.8 cm) long. The tube is heated with 1 in (2.5 cm) diameter ceramic band heaters. The leads of a 7-point thermocouple are distributed long the length of the tube, with some in the middle of the reactor zone (to measure gas temperature).

Reactor B: Schedule 80 Nickel 200 tube with an Inconel® 617 overlay, 18 in (45.7 cm) long, 1.5 in (3.8 cm) outer diameter, 0.84 in (2.1 cm) inner diameter. The reaction zone is 2 in (5.1 cm) long. The reactor zone is heated with an 8.5 in (21.6 cm) long×2.5 in (6.35 cm) split tube furnace. The leads of a 7-point thermocouple are distributed long the length of the tube, with some in the middle of the reactor zone (to measure gas temperature).

Reactor C: Hastelloy® C276 with gold lining. Length 5 in (12.7 cm)×0.50 in (1.3 cm) outer diameter×0.35 in (0.89 cm) inner diameter. The wall thickness is 0.15 in (3.8 mm). The thickness of the gold lining is 0.03 in (0.08 cm). The reactor zone is 2 in (5.1 cm) long and is heated with a ceramic band heater.

### Example 11

Reactor A (Inconel® 600 reaction surface) is used. The reactor inlet gas temperature (“Reactor Inlet T Gas” in Table 1) is the reaction temperature. Two runs are made at reaction temperatures of 724° C. and 725° C., respectively. In Run A, the reactant feed is undiluted with inert gas. In Run B, helium and reactant are fed in the ratio of 1.4:1. The benefit of the inert gas diluent is seen in the improved yield of Run B (80%) over that of Run A (71%). A lower concentration of fluorocarbon byproducts are made in Run B. Results are summarized in Table 11. Note that “sccm” in the table stands for “standard cubic centimeters per minute”.

TABLE 11

| | |
|---|---|
| A | B |

Reactor Conditions, Feeds

| | | |
|---|---|---|
| Preheat Control T setting | 700° C. | 700° C. |
| Preheat Gas T 1″ | 545° C. | 572° C. |
| Preheat Gas T 2″ | 635° C. | 655° C. |
| Preheat Gas T 3″ | 690° C. | 696° C. |
| Preheat Gas T 4″ | 718° C. | 720° C. |
| Reactor Control T setting | 700° C. | 700° C. |
| Reactor Inlet T wall | 711° C. | 710° C. |
| Reactor Middle T wall | 700° C. | 700° C. |
| Reactor Exit T wall | 622° C. | 623° C. |
| Reactor Inlet T gas | 724° C. | 725° C. |
| Reactor Middle T gas | 714° C. | 716° C. |
| Reactor Exit T gas | 675° C. | 673° C. |
| HFC-236fa Feed sccm | 25 sccm | 25 sccm |
| Helium Feed sccm | 0 sccm | 35 sccm |
| Residence Time in Reaction Zone | 42 | 18 |
| (seconds) | | |

Gas Chromatograph Results in Mole %

| | | |
|---|---|---|
| CHCF₃ (HFC-23) | 4.5 | 2.1 |
| CF₃CH═CF₂ (HFC-1225zc) | 51.6 | 47.7 |
| Octafluorocyclobutane (PFC-C318) | 1.8 | 2.0 |
| CF₃CH₂CF₃ (HFC-236fa) | 27.5 | 40.3 |
| C₄H₂F₆ (HFC-1336) | 1.8 | 0.7 |
| C₄HF₇ (HFC-1327) | 1.7 | 1.2 |
| C₄HF₉ (HFC-329) | 4.2 | 2.1 |
| Other | 3.1 | 2.1 |
| Unknown | 3.8 | 1.8 |
| Conversion (%) | 72.5 | 59.7 |
| Yield (%) | 71 | 80 |

### Example 12

Reactor A (Inconel® 600 reaction surface) is used in this study of the effect of temperature on conversion and yield. Run A is made at reactor temperature of 600° C. Runs B and C are made at 699° C. and 692° C., respectively. Runs A and B are diluted 4:1 with helium. Run C is undiluted. Run A (600° C.) conversion is low at 0.3%. Runs B and C (690-700° C.) have higher conversion, though still low compared to the conversion seen in Example 11, which was run at 725° C. and appreciably longer reaction zone residence times. Yields are reported, however are not reliable for such low conversions. The dependence of conversion on temperature and reaction zone residence time is plain from these experiments. Results are summarized in Table 12.

TABLE 12

| | | |
|---|---|---|
| A | B | C |

Reactor Conditions, Feeds

| | | | |
|---|---|---|---|
| Preheat Control T setting | 600 | 700 | 700 |
| (° C.) | | | |
| Preheat Gas T 1″ (° C.) | 417 | 497 | 443 |
| Preheat Gas T 2″ (° C.) | 511 | 604 | 546 |
| Preheat Gas T 3″ (° C.) | 563 | 660 | 623 |
| Preheat Gas T 4″ (° C.) | 592 | 691 | 676 |
| Reactor Control T setting | 601 | 700 | 700 |
| (° C.) | | | |
| Reactor Inlet T wall (° C.) | 615 | 718 | 722 |
| Reactor Middle T wall (° C.) | 601 | 700 | 700 |
| Reactor Exit T wall (° C.) | 566 | 661 | 653 |
| Reactor Inlet T gas (° C.) | 600 | 699 | 692 |
| Reactor Middle T gas (° C.) | 588 | 684 | 665 |
| Reactor Exit T gas (° C.) | 560 | 650 | 609 |
| Helium Feed sccm | 400 | 400 | 0 |
| HFC-236fa Feed sccm | 100 | 100 | 200 |
| Residence Time in Reaction | 2 | 2 | 5 |
| Zone (seconds) | | | |

Gas Chromatograph Results in Mole %

| | | | |
|---|---|---|---|
| CHCF₃ (HFC-23) | 0.0 | 0.0 | 0.1 |
| CHF═CF₂ (HFC-1123) | 0.0 | 0.0 | 0.1 |
| CF₃CH═CF₂ (HFC-1225zc) | 0.1 | 2.1 | 4.4 |
| CF₃CH₂CF₃ (HFC-236fa) | 99.7 | 97.6 | 95.3 |
| Other (<1%) | 0.2 | 0.2 | 0.3 |
| Conversion (%) | 0.3 | 2.4 | 4.7 |
| Yield (%) | 33 | 87.5 | 93.6 |

### Example 13

Reactor B (Nickel 200 reaction surface) is used. In this reactor the reactor temperature is the reactor center gas temperature (“Reactor Center Gas T” in Table 3). Runs A, B, and C are made at 800° C. with helium:reactant ratios of 0:1, 1:1, and 2:1, respectively. At these temperatures, higher than in Example 11, and at comparable reaction zone residence times, on the nickel surface, conversions are as high, and yields higher. In pyrolyses, higher temperatures generally lead to lower yields because of increased rates of undesirable side reactions giving unwanted byproducts. That this is not seen in Example 13 is testimony to the superiority of the nickel reaction surface to the nickel alloy reaction surface of Example 11. Further support for this conclusion is found in Run D, made at 850° C. with 4:1 helium dilution. Conversion is high at 76.9%, and the yield is 90.5%, the best of any of the Example 13 runs. Results are summarized in Table 13.

TABLE 13

| | | | |
|---|---|---|---|
| A | B | C | D |

Reactor Conditions, Feeds

| | | | | |
|---|---|---|---|---|
| Reactor Control T setting (° C.) | 839 | 834 | 832 | 885 |
| Reactor Inlet T wall (° C.) | 812 | 806 | 804 | 853 |
| Reactor Middle T wall (° C.) | 831 | 826 | 824 | 877 |
| Reactor Exit T wall (° C.) | 808 | 805 | 804 | 855 |
| Preheat Gas T 1″ (° C.) | 658 | 666 | 669 | 707 |
| Reactor Inlet gas T 2″ (° C.) | 736 | 740 | 741 | 786 |
| Reactor Inlet gas T 3″ (° C.) | 779 | 780 | 781 | 829 |
| Reactor Center gas T 4″ (° C.) | 800 | 800 | 800 | 850 |
| Reactor Exit gas T 5″ (° C.) | 800 | 800 | 799 | 851 |
| Reactor Exit gas T 6″ (° C.) | 776 | 777 | 777 | 829 |
| Exit gas T 7″ (° C.) | 738 | 741 | 740 | 791 |
| HFC-236fa Feed sccm | 200 | 200 | 200 | 200 |
| He Feed sccm | 0 | 200 | 400 | 800 |
| Residence Time in Reaction | 5 | 3 | 2 | 1 |
| Zone (seconds) | | | | |

GC Results in Mole %

| | | | | |
|---|---|---|---|---|
| CHCF₃ (HFC-23) | 4.1 | 2.5 | 2.0 | 2.5 |
| CHF═CF₂ (HFC-1123) | 0.7 | 1.0 | 1.0 | 1.4 |
| CF₃CH═CF₂ (HFC-1225zc) | 60.8 | 50.6 | 45.3 | 69.6 |
| CF₃CH₂CF₃ (HFC-236fa) | 28.2 | 37.7 | 50.2 | 23.1 |
| C₄H₂F₆ (HFC-1336) | 1.3 | 0.6 | 0.4 | 0.0 |
| Other (<1% produced) | 2.1 | 1.0 | 1.1 | 2.1 |
| Unknown | 2.8 | 6.6 | 0.0 | 1.2 |
| Conversion (%) | 71.8 | 62.3 | 49.8 | 76.9 |
| Yield (%) | 84.7 | 81.2 | 90.9 | 90.5 |

### Example 14

Reactor C (gold reaction surface). Like nickel, the gold surface gives high yields and therefore reduced side reactions producing unwanted byproducts. The inert gas diluent effect (reduction) on conversion is less on gold than on nickel or nickel alloy surfaces. At 800° C. (Runs A and B) conversions are lower than those of Runs B and C of Example 13 but the average yield is higher. Results are summarized in Table 14.

TABLE 14
Reactor Conditions, Feeds

| | A | B | C | D | E | F |
|---|---|---|---|---|---|---|
| Reactor Temp (° C.) | 800 | 800 | 700 | 700 | 600 | 600 |
| He Feed sccm | 15 | 20 | 15 | 20 | 15 | 20 |
| HFC-236fa Feed sccm | 10 | 5 | 10 | 5 | 10 | 5 |
| Residence Time in | 8 | 8 | 8 | 8 | 8 | 8 |
| Reaction Zone | | | | | | |
| (seconds) | | | | | | |

GC Mole %

| | | | | | | |
|---|---|---|---|---|---|---|
| CHF₃ and CH₂F₂ | 1.9 | 1.9 | 0.1 | 0.1 | ND* | ND |
| CHF═CF₂ (HFC-1123) | 0.8 | 0.9 | ND | ND | ND | ND |
| CF₃CH₃ (HFC-143a) | 0.2 | 0.2 | ND | ND | ND | ND |
| CF₃CH═CF₂ (HFC- | 33.3 | 36.6 | 1.7 | 1.8 | 0.2 | 0.1 |
| 1225zc) | | | | | | |
| CF₃CHFCF₃ (HFC- | 0.2 | 0.2 | 0.1 | 0.1 | 0.1 | 0.1 |
| 227ea) | | | | | | |
| CF₃CH₂CF₃ (HFC-236fa) | 61.9 | 58.4 | 97.6 | 97.6 | 99.4 | 99.5 |
| Unknown | 0.6 | 0.5 | 0.3 | 0.1 | 0.2 | 0.1 |
| Conversion (%) | 38.1 | 41.6 | 2.4 | 2.4 | 0.6 | 0.5 |
| Yield (%) | 87.4 | 88.0 | 71 | 75 | 33 | 20 |

*ND = not detected

Examples 11-14 show the specificity of the pyrolysis according to this invention, which gives the product CF₃CH═CF₂ in good yield at good conversion with only small amounts of unwanted byproducts. Nickel is superior to nickel alloy as the reaction surface in giving higher yields of product. Gold is superior to nickel.

Conversions are low up to about 700° C., being good at 725° C. and above with no deterioration in performance even at 850° C.

What is claimed is:

1. An azeotrope or near-azeotrope composition comprising from about 57.8 mole percent to about 82.0 mole percent HFC-1225zc and hydrogen fluoride.

2. The azeotrope or near-azeotrope composition of claim 1 comprising from about 57.8 mole percent to about 82.0 mole percent HFC-1225zc and from about 42.2 mole percent to about 18.0 mole percent hydrogen fluoride.

3. The azeotrope or near-azeotrope composition of claim 1 comprising from about 57.8 mole percent to about 82.0 mole percent HFC-1225zc and from about 42.2 mole percent to about 18.0 mole percent hydrogen fluoride, wherein the vapor pressure is from about 17.4 psi (120 kPa) to about 745 psi (5137 kPa) at a temperature of from about −20° C. to about 100° C.

4. The azeotrope or near-azeotrope composition of claim 1 wherein said composition consists essentially of from about 57.8 mole percent to about 82.0 mole percent HFC-1225zc and from about 42.2 mole percent to about 18.0 mole percent hydrogen fluoride, wherein the vapor pressure is from about 17.4 psi (120 kPa) to about 745 psi (5137 kPa) at a temperature of from about −20° C. to about 100° C.

5. The azeotrope composition of claim 1 comprising from about 64.5 mole percent to about 69.0 mole percent HFC-1225zc and from about 35.5 mole percent to about 31.0 mole percent hydrogen fluoride, wherein the vapor pressure is from about 17.4 psi (120 kPa) to about 745 psi (5137 kPa) at a temperature of from about −20° C. to about 100° C.

6. The azeotrope composition of claim 1 wherein said composition consists essentially of from about 64.5 mole percent to about 69.0 mole percent HFC-1225zc and from about 35.5 mole percent to about 31.0 mole percent hydrogen fluoride, wherein the vapor pressure is from about 17.4 psi (120 kPa) to about 745 psi (5137 kPa) at a temperature of from about −20° C. to about 100° C.

7. The azeotrope or near-azeotrope composition of claim 1, wherein said composition is characterized by a difference between dew point pressure and bubble point pressure that is less than or equal to 3%, based upon bubble point pressure.

8. A process for the separation of HFC-1225zc from HFC-236fa comprising:
(a) forming a mixture of HFC-1225zc, HFC-236fa, and hydrogen fluoride; and
(b) subjecting said mixture to a distillation step forming a column distillate composition comprising an azeotrope or near-azeotrope composition of hydrogen fluoride and HFC-1225zc essentially free of HFC-236fa.

9. The process of claim 8 wherein said distillation step further forms a column-bottoms composition comprising HFC-236fa.

10. The process of claim 9 wherein said column-bottoms composition comprises HFC-236fa essentially free of hydrogen fluoride.

11. The process of claim 8 wherein said mixture of HFC-1225zc, HFC-236fa, and hydrogen fluoride comprises equimolar quantities of each component.

12. The process of claim 8 wherein said mixture of HFC-1225zc, HFC-236fa, and hydrogen fluoride comprises excess HFC-1225zc.

13. A process for the separation of HFC-1225zc from a mixture comprising an azeotrope or near-azeotrope composition of HFC-1225zc and hydrogen fluoride, said process comprising:
a) subjecting said mixture to a first distillation step in which a composition enriched in either (i) hydrogen fluoride or (ii) HFC-1225zc is removed as a first distillate composition with a first bottoms composition being enriched in the other of said components (i) or (ii); and
b) subjecting said first distillate composition to a second distillation step conducted at a different pressure in which the component enriched as first bottoms composition in (a) is removed in a second distillate composition with a second bottoms composition enriched in the same component which was enriched in the first distillate composition.

14. The process of claim 13 wherein said first bottoms composition comprises HFC-1225zc essentially free of hydrogen fluoride.

15. The process of claim 13 wherein said second bottoms composition comprises hydrogen fluoride essentially free of HFC-1225zc.

16. The process of claim 13 wherein said first distillation step is carried out at a pressure greater than the pressure of the second distillation step.

17. The process of claim 13 wherein said mixture consists essentially of HFC-1225zc in combination with an effective amount of hydrogen fluoride to form an azeotrope or near-azeotrope composition with hydrogen fluoride, said azeotrope or near-azeotrope composition containing from about 57.8 mole percent to about 82.0 mole percent HFC-1225zc.

18. A process for the purification of HFC-1225zc from a mixture of HFC-1225zc, HFC-236fa, and hydrogen fluoride, said process comprising:
a) subjecting said mixture to a first distillation step to form a first distillate comprising an azeotrope or near-azeotrope composition containing HFC-1225zc and hydrogen fluoride and a first bottoms comprising HFC-236fa;
b) subjecting said first distillate to a second distillation step from which a composition enriched in either (i) hydrogen fluoride or (ii) HFC-1225zc is removed as a second distillate composition with a second bottoms composition being enriched in the other of said components (i) or (ii); and
c) subjecting said second distillate composition to a third distillation step conducted at a different pressure than the second distillation step in which the component enriched in the second bottoms composition in (b) is removed in a third distillate composition with a third bottoms composition enriched in the same component that was enriched in the second distillate composition.

19. A process to produce HFC-1225zc comprising:
a) feeding HFC-236fa to a reaction zone for dehydrofluorination to form a reaction product composition comprising HFC-1225zc, unreacted HFC-236fa and hydrogen fluoride;
b) subjecting said reaction product composition to a first distillation step to form a first distillate composition comprising an azeotrope or near-azeotrope composition containing HFC-1225zc and hydrogen fluoride and a first bottoms composition comprising HFC-236fa;
c) subjecting said first distillate composition to a second distillation step from which a composition enriched in either (i) hydrogen fluoride or (ii) HFC-1225zc is removed as a second distillate composition with a second bottoms composition being enriched in the other of said components (i) or (ii); and
d) subjecting said second distillate composition to a third distillation step conducted at a different pressure than the second distillation step in which the component enriched in the second bottoms composition in (c) is removed in a third distillate composition with a third bottoms composition enriched in the same component that was enriched in the second distillate composition.

20. The process of claim 19, further comprising recycling at least some portion of said first bottoms composition to said reaction zone.

21. The process of claim 19, further comprising recycling at least some portion of said second bottoms composition or said third bottoms composition to said reaction zone.

22. The process of claim 19, further comprising recovering at least some portion of said second bottoms composition or third bottoms composition as HFC-1225zc essentially free of HFC-236fa and hydrogen fluoride.

23. The process of claim 19, further comprising recycling at least some portion of said second bottoms composition or said third bottoms composition to said first distillation step.

24. A process for the separation of HFC-236fa from a mixture comprising an azeotrope or near-azeotrope composition of HFC-236fa and hydrogen fluoride, said process comprising:
a) subjecting said mixture to a first distillation step in which a composition enriched in either (i) hydrogen fluoride or (ii) HFC-236fa is removed as a first distillate composition with a first bottoms composition being enriched in the other of said components (i) or (ii); and
b) subjecting said first distillate composition to a second distillation step conducted at a different pressure in which the component enriched as first bottoms composition in (a) is removed in a second distillate composition with a second bottoms composition enriched in the same component which was enriched in the first distillate composition.

25. The process of claim 24 wherein said first bottoms composition comprises HFC-236fa essentially free of hydrogen fluoride.

26. The process of claim 24 wherein said second bottoms composition comprises hydrogen fluoride essentially free of HFC-236fa.

27. The process of claim 24 wherein said second distillation step is carried out at a pressure greater than the pressure of the first distillation step.
